(19) United States
(12) Patent Application Publication
(10) Pub. No.: US 2026/0070989 A1
(43) Pub. Date: Mar. 12, 2026

(54) STABLE LYOPHILIZED FORMULATION OF AN ANTI-alpha4beta7 ANTIBODY

(71) Applicant: INTAS PHARMACEUTICALS LTD., Ahmedabad, Gujarat (IN)

(72) Inventors: Kinjal INGARODIYA, Ahmedabad (IN); Rahul JANGLE, Ahmedabad (IN); Pravin A. NAIR, Ahmedabad (IN)

(21) Appl. No.: 18/704,330

(22) PCT Filed: Oct. 12, 2022
(86) PCT No.: PCT/IB2022/059777

§ 371 Date: Apr. 24, 2024

(30) Foreign Application Priority Data
Oct. 29, 2021 (IN) 202121049583

(51) Int. Cl. C07K 16/28

(52) U.S. Cl. CPC C07K 16/2839; C07K 2317/24

(57) ABSTRACT

The invention provides stable lyophilized formulation of anti-α4ß7 antibody comprising buffer, sugar, bulking agent, viscosity reducer, antioxidant and surfactant.

### RELATED APPLICATIONS

This application is related to Indian Provisional Application IN202121049583 filed 29 Oct. 2021 and is incorporated herein in its entirety.

### FIELD OF THE INVENTION

The present invention relates to stable lyophilized pharmaceutical formulation of an anti-α4ß7 antibody

### BACKGROUND OF THE INVENTION

Over the past two decades, recombinant DNA technology has led to the commercialization of many proteins, particularly antibody therapeutics. The effectiveness of these therapeutic antibodies is majorly dependent on the stability, route of administration and their dosage forms and concentrations. This in turn, necessitates therapeutic antibodies to be formulated appropriately to retain the stability and activity of a therapeutic antibody.

Formulations for each route of administration and dosage forms may be unique and, therefore, have specific requirements. Solid dosage forms, such as lyophilized powders, are generally more stable than liquid (aqueous) formulations. However, reconstitution of the lyophilized formulation requires a significant vial overfill, care in handling and involves high production cost relative to a liquid formulation. While liquid formulations are advantageous in these and are usually preferred for injectable protein therapeutics (in terms of convenience for the end user and ease of preparation for the manufacturer), this form may not always be feasible given the susceptibility of proteins to denaturation, aggregation and oxidation under stresses such as temperature, pH changes, agitation etc., All of these stress factors could result in the loss of biological activity of a therapeutic protein/antibody. In particular, high concentration liquid formulations are susceptible to degradation and/or aggregation. Nevertheless, high concentration formulations may be desirable for subcutaneous or intravenous route of administration, as the frequency of administration and injection volume is reduced. On the other hand, specific treatment schedule and dosing might require a low concentration formulation and prefer intravenous route of administration for more predictable delivery and complete bioavailability of the therapeutic drug.

Hence, designing a formulation that is stable at high or low concentrations of the therapeutic protein/antibody, aiding in different route of administration (intravenous or subcutaneous) and which is suitable in lyophilized or liquid form, pose a significant developmental challenge. Further, every protein or antibody with its unique characteristics and properties of degradation, adds to the complexity in the development of a stable formulation and may demand a specific formulation.

### OBJECTS OF THE INVENTION

The main object of the present invention is to provide stable lyophilized pharmaceutical formulation of an anti-α4ß7 antibody comprising buffer, sugar, bulking agent, viscosity reducer, antioxidant and surfactant.

Another object of the present invention is to provide stable lyophilized pharmaceutical formulation of an anti-α4ß7 antibody comprising buffer, sugar, bulking agent, viscosity reducer, antioxidant and surfactant. Wherein, wherein the molar ratio of sucrose to anti-α4ß7 antibody (mole:mole) is less than 600:1.

Another object of the present invention is to provide stable lyophilized pharmaceutical formulation of an anti-α4ß7 antibody comprising buffer, sugar, bulking agent, viscosity reducer, antioxidant, surfactant and at pH about 6.0 to 7.0.

Another object of the present invention is to provide stable lyophilized pharmaceutical formulation of an anti-α4ß7 antibody comprising glycine as a bulking agent, L-lysine HCl as a viscosity reducer and L-methionine as an antioxidant and at pH about 6.0 to 7.0.

Another object of the present invention is to provide stable lyophilized pharmaceutical formulation of an anti-α4ß7 antibody comprising histidine, histidine HCl, sucrose, glycine, L-lysine HCl, L-methionine and at pH about 6.0 to 7.0, wherein the molar ratio of sucrose to anti-α4β7 antibody (mole:mole) is less than 600:1.

Another object the present invention is to provide stable lyophilized pharmaceutical formulation of an anti-α4ß7 antibody comprising buffer, sugar, bulking agent, viscosity reducer, antioxidant and surfactant; wherein α4ß7 antibody is at a concentration of about 50-200 mg/mL, buffer is at a concentration range of 0-100 mM, sugar is at a concentration range of 0-100 mg/mL, bulking agent is at a concentration range of 0-50 mg/mL, viscosity reducer is at a concentration range of 0-10 mg/mL, antioxidant is at a concentration range of 0-2 mg/mL, surfactant is at a concentration range of 0-1 mg/mL and at pH about 6.0 to 7.0.

Another object the present invention is to provide stable lyophilized pharmaceutical formulation of an anti-α4ß7 antibody comprising glycine as a bulking agent, L-lysine HCl as a viscosity reducer and L-methionine as an antioxidant; wherein α4ß7 antibody is at concentration of about 50-200 mg/mL, glycine is in the concentration range of 0-50 mg/mL, L-lysine HCl is in the concentration range of 0-10 mg/mL, L-methionine is in the concentration range of 0-2 mg/mL and at pH about 6.0 to 7.0.

Another object of the present invention is provide stable lyophilized pharmaceutical formulation of an anti-α4ß7 antibody comprising 0-100 mM histidine/histidine HCl, 0-100 mg/mL sucrose, 0-50 mg/mL glycine, 0-10 mg/mL L-lysine HCl, 0-2 mg/mL L-methionine, 0-1 mg/mL polysorbate 80 and at pH about 6.0 to 7.0.

Another object of the present invention is to provide stable lyophilized pharmaceutical formulation of Vedolizumab comprising 50 mM histidine/histidine HCl, 80 mg/mL sucrose, 15 mg/mL glycine, 4 mg/mL L-lysine HCl, 0.75 mg/mL L-methionine, 0.6 mg/mL polysorbate 80 at pH 6.3, wherein the molar ratio of Sucrose to Vedolizumab (mole:mole) is less than 600:1.

Another object of the present invention is to provide stable lyophilized pharmaceutical formulation of Vedolizumab comprising 50 mM histidine/histidine HCl, 80 mg/mL sucrose, 30 mg/ml glycine, 8 mg/mL L-lysine HCl, 0.75 mg/mL L-methionine, 0.6 mg/mL polysorbate 80 at pH 6.3, wherein the molar ratio of Sucrose to Vedolizumab (mole:mole) is less than 600:1.

### SUMMARY OF THE INVENTION

The main aspect of the present invention is to provide stable lyophilized pharmaceutical formulation of an anti-α4ß7 antibody comprising buffer, sugar, bulking agent, viscosity reducer, antioxidant and surfactant.

Another aspect of the present invention is to provide stable lyophilized pharmaceutical formulation of an anti-α4ß7 antibody comprising buffer, sugar, bulking agent, viscosity reducer, antioxidant and surfactant. Wherein, wherein the molar ratio of sucrose to anti-α4β7 antibody (mole:mole) is less than 600:1.

Another aspect of the present invention is to provide stable lyophilized pharmaceutical formulation of an anti-α4ß7 antibody comprising buffer, sugar, bulking agent, viscosity reducer, antioxidant, surfactant and at pH about 6.0 to 7.0.

Another aspect of the present invention is to provide stable lyophilized pharmaceutical formulation of an anti-α4ß7 antibody comprising glycine as a bulking agent, L-lysine HCl as a viscosity reducer and L-methionine as an antioxidant and at pH about 6.0 to 7.0.

Another aspect of the present invention is to provide stable lyophilized pharmaceutical formulation of an anti-α4ß7 antibody comprising histidine, histidine HCl, sucrose, glycine, L-lysine HCl, L-methionine and at pH about 6.0 to 7.0, wherein the molar ratio of sucrose to anti-α4β7 antibody (mole:mole) is less than 600:1.

Another aspect the present invention is to provide stable lyophilized pharmaceutical formulation of an anti-α4ß7 antibody comprising buffer, sugar, bulking agent, viscosity reducer, antioxidant and surfactant; wherein α4ß7 antibody is at a concentration of about 50-200 mg/mL, buffer is at a concentration range of 0-100 mM, sugar is at a concentration range of 0-100 mg/mL, bulking agent is at a concentration range of 0-50 mg/mL, viscosity reducer is at a concentration range of 0-10 mg/mL, antioxidant is at a concentration range of 0-2 mg/mL, surfactant is at a concentration range of 0-1 mg/mL and at pH about 6.0 to 7.0.

Another aspect the present invention is to provide stable lyophilized pharmaceutical formulation of an anti-α4ß7 antibody comprising glycine as a bulking agent, L-lysine HCl as a viscosity reducer and L-methionine as an antioxidant; wherein α4ß7 antibody is at concentration of about 50-200 mg/mL, glycine is in the concentration range of 0-50 mg/mL, L-lysine HCl is in the concentration range of 0-10 mg/mL, L-methionine is in the concentration range of 0-2 mg/mL and at pH about 6.0 to 7.0.

Another aspect of the present invention is to provide stable lyophilized pharmaceutical formulation of an anti-α4ß7 antibody comprising 0-100 mM histidine/histidine HCl, 0-100 mg/mL sucrose, 0-50 mg/mL glycine, 0-10 mg/mL L-lysine HCl, 0-2 mg/mL L-methionine, 0-1 mg/mL polysorbate 80 and at pH about 6.0 to 7.0.

Another aspect of the present invention is to provide stable lyophilized pharmaceutical formulation of Vedolizumab comprising 50 mM histidine/histidine HCl, 80 mg/mL sucrose, 15 mg/ml glycine, 4 mg/mL L-lysine HCl, 0.75 mg/mL L-methionine, 0.6 mg/mL polysorbate 80 at pH 6.3, wherein the molar ratio of Sucrose to Vedolizumab (mole:mole) is less than 600:1.

Another aspect of the present invention is to provide stable lyophilized pharmaceutical formulation of Vedolizumab comprising 50 mM histidine/histidine HCl, 80 mg/mL sucrose, 30 mg/ml glycine, 8 mg/mL L-lysine HCl, 0.75 mg/mL L-methionine, 0.6 mg/mL polysorbate 80 at pH 6.3, wherein the molar ratio of Sucrose to Vedolizumab (mole:mole) is less than 600:1.

### BRIEF DESCRIPTION OF DRAWING

In order that disclosure may be readily understood and put into practical effect, reference will now be made to exemplary embodiments as illustrated with reference to the accompanying figures. The figure with a detailed description below, are incorporated in and form part of the specification, and serve to further illustrate the embodiments and explain various principles and advantages, in accordance with the present disclosure wherein:

FIG. 1: Percentage purity (CEX) trend analysis of RMP and F1 to F11 at 40° C.

FIG. 2: Percentage purity (SEC) trend analysis of RMP and F1 to F11 at 40° C.

FIG. 3: CEX trend analysis of batch 1, 2 & 3 at real time condition (5° C.±3° C.).

FIG. 4: SEC trend analysis of batch 1, 2 & 3 at real time condition (5° C.±3° C.).

FIG. 5: Relative potency trend analysis of batch 1, 2 & 3 at real time condition (5° C.±3° C.).

FIG. 6: pH trend analysis of batch 1, 2 & 3 and RMP at accelerated time condition (25° C.±2° C.).

FIG. 7: pH trend analysis of batch 1, 2 & 3 and RMP at stress stability condition (40° C.±2° C.).

FIG. 8: CEX trend analysis of batch 1, 2 & 3 and RMP at accelerated time condition (25° C.±2° C.).

FIG. 9: CEX trend analysis of batch 1, 2 & 3 and RMP at stress stability condition (40° C.±2° C.).

FIG. 10: SEC trend analysis of Batch 1, 2 & 3 and RMP at accelerated time condition (25° C.±2° C.).

FIG. 11: SEC trend analysis of batch 1, 2 & 3 and RMP at stress stability condition (40° C.±2° C.).

FIG. 12: Relative potency trend analysis of Batch 1, 2 & 3 and RMP at accelerated time condition (25° C.±2° C.).

FIG. 13: Relative potency trend analysis of batch 1, 2 & 3 and RMP at stress stability condition (40° C.±2° C.).

### DETAILED DESCRIPTION OF THE INVENTION

### Definition

The following definitions are provided to facilitate understanding of certain terms used throughout the specification.

Unless defined otherwise, all technical and scientific terms used herein have the same meaning as commonly understood by those of ordinary skill in the art to which the invention belongs. Although any methods and materials similar or equivalent to those described herein can be used in the practice or testing of particular embodiments, preferred embodiments of compositions, methods and materials are described herein. For the purposes of the present disclosure, the following terms are defined below.

The articles “a,” “an,” and “the” are used herein to refer to one or to more than one (i.e., to at least one, or to one or more) of the grammatical object of the article. By way of example, “an element” means one element or one or more elements.

The words “comprise”, “comprises”, and “comprising” are to be interpreted inclusively rather than exclusively. The words “consist”, “consisting”, and its variants, are to be interpreted exclusively, rather than inclusively. While various embodiments in the specification are presented using “comprising” language, under other circumstances, a related embodiment is also intended to be interpreted and described using “consisting of” or “consisting essentially of” language.

The term “pharmaceutical formulation” refers to a preparation that contains an anti-α4ß7 antibody in such form as to permit the biological activity of the antibody to be effective, and which contains no additional components which are unacceptably toxic to a subject to which the formulation would be administered.

A “stable” formulation is one in which the antibody therein substantially retains its physical stability and/or chemical stability and/or its biological activity upon storage. In one aspect, the formulation substantially retains its physical and chemical stability, as well as its biological activity upon storage. The storage period is generally selected based on the intended shelf-life of the formulation. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10:29-90 (1993), for example.

Stability studies provides evidence of the quality of an antibody under the influence of various environmental factors during the course of time. ICH's “Q1A: Stability Testing of New Drug Substances and Products,” states that data from accelerated stability studies can be used to evaluate the effect of short-term excursions higher or lower than label storage conditions that may occur during the shipping of the antibodies.

Various analytical methods are available for measuring the physical and chemical degradation of the antibody in the pharmaceutical formulations. An antibody “retains its physical stability” in a pharmaceutical formulation if it shows substantially no signs of aggregation, precipitation and/or denaturation upon visual examination of color and/or clarity, or as measured by UV light scattering or by size exclusion chromatography. An antibody is said to “retain its chemical stability” in a pharmaceutical formulation when its shows no or minimal formation of product variants which may include variants as a result of chemical modification of antibody of interest such as deamination, oxidation etc. Analytical methods such as ion exchange chromatography and hydrophobic ion chromatography may be used to investigate the chemical product variants.

As used herein, “buffering agent” refers to a buffer that resists changes in pH by the action of its acid-base conjugate components. The buffering agent may be present in a liquid or solid formulation of the invention. The buffering agent adjusts the pH of the formulation to about 5.0 to about 7.5, to about 5.5 to about 7.5, to about 6.0 to about 6.5, or to a pH of about 6.3. In one aspect, examples of buffering agents that will control the pH in the 5.0 to 7.5 range include acetate, succinate, gluconate, histidine, citrate, phosphate, maleate, cacodylate.

A “histidine buffer” is a buffer comprising histidine ions. Examples of histidine buffers include histidine chloride, histidine acetate, histidine phosphate, histidine sulfate solutions. The histidine buffer or histidine-HCl buffer has a pH between about pH 5.5 to 6.5, about pH 6.1 to 6.5, or about pH 6.3.

A “surfactant” herein refers to an agent that lowers surface tension of a liquid. The surfactant can be a nonionic surfactant. In one aspect, examples of surfactants herein include polysorbate (polyoxyethylene sorbitan monolaurate, for example, polysorbate 20 and, polysorbate 80).

Molar amounts and ratios of anti-α4ß7 antibody to other excipients described herein are calculated on the assumption of an approximate molecular weight of about 150,000 daltons for the antibody.

The actual antibody molecular weight may differ from 150,000 daltons, depending on amino acid composition or post-translational modification, e.g., as dependent on the cell line used to express the antibody. Actual antibody molecular weight can be ±5% of 150,000 daltons.

An ‘anti-α4ß7 antibody’ herein refers to Vedolizumab antibody.

The main embodiment of the present invention is to provide stable lyophilized pharmaceutical formulation of an anti-α4ß7 antibody comprising buffer, sugar, bulking agent, viscosity reducer, antioxidant and surfactant.

Another embodiment of the present invention is to provide stable lyophilized pharmaceutical formulation of an anti-α4ß7 antibody comprising buffer, sugar, bulking agent, viscosity reducer, antioxidant and surfactant. Wherein, wherein the molar ratio of sucrose to anti-α4β7 antibody (mole:mole) is less than 600:1.

Another embodiment of the present invention is to provide stable lyophilized pharmaceutical formulation of an anti-α4ß7 antibody comprising buffer, sugar, bulking agent, viscosity reducer, antioxidant, surfactant and at pH about 6.0 to 7.0.

Another embodiment of the present invention is provide stable lyophilized pharmaceutical formulation of an anti-α4ß7 antibody comprising glycine as a bulking agent, L-lysine HCl as a viscosity reducer and L-methionine as an antioxidant and at pH about 6.0 to 7.0.

Another embodiment of the present invention is provide stable lyophilized pharmaceutical formulation of an anti-α4ß7 antibody comprising histidine, histidine HCl, sucrose, glycine, L-lysine HCl, L-methionine and at pH about 6.0 to 7.0, wherein the molar ratio of sucrose to anti-α4β7 antibody (mole:mole) is less than 600:1. Preferably, 573:1

Another embodiment the present invention is to provide stable lyophilized pharmaceutical formulation of an anti-α4ß7 antibody comprising buffer, sugar, bulking agent, viscosity reducer, antioxidant and surfactant; wherein α4ß7 antibody is at a concentration of about 50-200 mg/mL, buffer is at a concentration range of 0-100 mM, sugar is at a concentration range of 0-100 mg/mL, bulking agent is at a concentration range of 0-50 mg/mL, viscosity reducer is at a concentration range of 0-10 mg/mL, antioxidant is at a concentration range of 0-2 mg/mL, surfactant is at a concentration range of 0-1 mg/mL and at pH about 6.0 to 7.0.

In another embodiment of the present invention histidine and histidine HCl is used as buffer in the formulation of anti-α4ß7 antibody. Wherein, buffer is at a concentration range of 0-100 mM. Preferably, buffer has concentration of 50 mM.

In another embodiment of the present invention, sucrose is used as a sugar in the formulation of anti-α4ß7 antibody. Wherein, sugar is a concentration range of 0-100 mg/mL. Preferably, sugar has concentration 70-90 mg/mL. More preferably sugar has concentration 80 mg/ml.

In another embodiment of the present invention, glycine is used as bulking agent in the formulation of anti-α4ß7 antibody. Wherein bulking agent is at a concentration range of 0-50 mg/mL. Preferably, bulking agent has concentration of about 15-30 mg/mL.

In yet another embodiment of the present invention, L-lysine HCl is used as viscosity reducer in the formulation of anti-α4ß7 antibody. Wherein viscosity reducer is at a concentration range of 0-10 mg/mL. Preferably, viscosity reducer has concentration of about 4-8 mg/mL.

In another embodiment of the present invention, polysorbate 80 is used as surfactant. Wherein surfactant is at a concentration range of 0-1 mg/mL. Preferably, surfactant has concentration of about 0.6 mg/mL.

In another embodiment of the present invention, L-methionine is used as an antioxidant in the formulation of anti-α4ß7 antibody. Wherein, antioxidant is at a concentration range of 0-2 mg/mL. Preferably, antioxidant has concentration of about 0.75 mg/mL.

In another embodiment of the present invention, stable lyophilized pharmaceutical formulation of an anti-α4ß7 antibody pH is in the range of about 6.0 to 7.0. Preferably, stable lyophilized pharmaceutical formulation of an anti-α4ß7 antibody formulation has pH 6.3.

In another embodiment of the present invention formulation has anti-α4ß7 antibody is at concentration of about 50-200 mg/mL. Preferably, 60 mg/mL.

In another embodiment of the present invention stable lyophilized pharmaceutical formulation of an anti-α4ß7 antibody is for intravenous administration.

Another embodiment the present invention is to provide stable lyophilized pharmaceutical formulation of an anti-α4ß7 antibody comprising glycine as a bulking agent, L-lysine HCl as a viscosity reducer and L-methionine as an antioxidant; wherein α4ß7 antibody is at concentration of about 50-200 mg/mL, glycine is in the concentration range of 0-50 mg/mL, L-lysine HCl is in the concentration range of 0-10 mg/mL, L-methionine is in the concentration range of 0-2 mg/mL, and at pH about 6.0 to 7.0.

Another embodiment of the present invention is to provide stable lyophilized pharmaceutical formulation of an anti-α4ß7 antibody comprising 0-100 mM histidine/histidine HCl, 0-100 mg/mL sucrose, 0-50 mg/mL glycine, 0-10 mg/mL L-lysine HCl, 0-2 mg/mL L-methionine, 0-1 mg/ml polysorbate 80 and at pH about 6.0 to 7.0.

Another embodiment of the present invention is to provide stable lyophilized pharmaceutical formulation of Vedolizumab comprising 50 mM histidine/histidine HCl, 80 mg/mL sucrose, 15 mg/mL glycine, 4 mg/mL L-lysine HCl, 0.75 mg/mL L-methionine, 0.6 mg/mL polysorbate 80 at pH 6.3, wherein the molar ratio of Sucrose to Vedolizumab (mole:mole) is less than 600:1.

Another embodiment of the present invention is to provide stable lyophilized pharmaceutical formulation of Vedolizumab comprising 50 mM histidine/histidine HCl, 80 mg/mL sucrose, 30 mg/mL glycine, 8 mg/mL L-lysine HCl, 0.75 mg/mL L-methionine, 0.6 mg/mL polysorbate 80 at pH 6.3, wherein the molar ratio of Sucrose to Vedolizumab (mole:mole) is less than 600:1.

In another embodiment of the present invention is to provide stable lyophilized pharmaceutical formulation of Vedolizumab wherein ratio of Sugar to Vedolizumab (mole:mole) is less than 600:1. In another aspect, the ratio of Sugar to Vedolizumab (mole:mole) is at least about 575:1; at least about 550:1 or at least about 500:1.

The embodiments of the present invention are further described using specific examples herein after. The examples are provided for better understanding of certain embodiments of the invention and not, in any manner, to limit the scope thereof. Possible modifications and equivalents apparent to those skilled in the art using the teachings of the present description and the general art in the field of the invention shall also from the part of this specification and are intended to be included within the scope of it.

### EXAMPLES

### Example 1: Screening of Formulation Components

An anti-α4ß antibody, Vedolizumab, suitable for storage in the present pharmaceutical composition is produced by standard methods known in the art. For example, Vedolizumab is prepared by recombinant expression of immunoglobulin light and heavy chain genes in a mammalian host cell such as Chinese Hamster Ovary cells. Further, the expressed Vedolizumab is harvested and the crude harvest is subjected to standard downstream process steps that include purification, filtration and optionally dilution or concentration steps. For example, the crude harvest of Vedolizumab may be purified using standard chromatography techniques such as affinity chromatography, ion-exchange chromatography and combinations thereof. The purified Vedolizumab solution can additionally be subjected to one or more filtration steps, and the solution obtained is subjected to further formulation studies.

Method of Preparation: Vedolizumab formulation was prepared in composition given in the Table 1 by dissolving the excipients in water for injection. The protein concentration was set to 60 mg/mL and the pH of the formulation is set to 6.3 similar to the reference formulation (ENTYVIO®). 5.52 mL of drug product solution is filled in 20 mL USP type 1 glass vial & half stoppered with coated bromobutyl rubber stopper for lyophilization process. Vials are stoppered & sealed with aluminum seal. Formulations were charged for stability at 40° C. for 28 days.

Different excipients with different concentration were tested. Total 11 formulations (F1 to F11) were prepared with different concentration of excipients as shown in below Table 1.

TABLE 1
Composition of Formulation F1 to F11

| | | Lyophilized Formulation | | | | | | | | | | | |
|---|---|---|---|---|---|---|---|---|---|---|---|---|---|
| Components | Ingredient | F1 | F2 | F3 | F4 | F5 | F6 | F7 | F8 | F9 | F10 | F11 | RMP |
| Active | Vedolizumab | 60.0 mg/mL | | | | | | | | | | | 60 |
| Protein | | | | | | | | | | | | | mg/mL |
| Buffer | L- Histidine | 4.59 mg/mL | | | | | | | | | | | 4.60 |
| | | | | | | | | | | | | | mg/mL |
| | L- Histidine | 4.28 mg/mL | | | | | | | | | | | 4.28 |
| | monohydrochloride | | | | | | | | | | | | mg/mL |
| Stabilizer | Sucrose | 80 mg/mL (ratio of sugar to protein is 573) | | | | | | | | | | | 100 |
| | | | | | | | | | | | | | mg/mL |
| | | | | | | | | | | | | | (ratio of |
| | | | | | | | | | | | | | sugar to |
| | | | | | | | | | | | | | protein |
| | | | | | | | | | | | | | is 715) |
| | L-Arginine HCl | — | | | | | | | | | | | 26.34 |
| | | | | | | | | | | | | | mg/mL |
| Bulking | Glycine | — | 30 | — | — | — | 30 | 30 | — | 30 | 15 | 30 | — |
| Agent | | | mg/mL | | | | mg/mL | mg/mL | | mg/mL | mg/mL | mg/mL | |
| | Ratio of bulking | — | 400 | — | — | — | 400 | 400 | — | 400 | 200 | 400 | — |
| | agent to protein | | | | | | | | | | | | |
| Antioxidant | L- Methionine | — | — | 1.8 | — | 1.49 | — | 1.49 | 1.49 | 1.49 | 0.75 | 0.75 | — |
| | | | | mg/mL | | mg/mL | | mg/mL | mg/mL | mg/mL | mg/mL | mg/mL | |
| Viscosity | L- Lysine HCl | — | — | — | 8 | — | 8 | — | 8 | 8 | 4 | 8 | — |
| Reducer | | | | | mg/mL | | mg/mL | | mg/mL | mg/mL | mg/mL | mg/mL | |

| | | | |
|---|---|---|---|
| Nonionic | Polysorbate 80 | 0.6 mg/mL | 0.6 |
| Surfactant | | | mg/mL |
| Vehicle | Water for injection | Q.S. to 1 mL | Q.S to 1 mL |
| pH | pH 6.3 | pH 6.3 | pH 6.3 |

The Vedolizumab lyophilized drug product was formulated in above mention different buffers and stability studies (40° C.±2° C.) were performed and all the formulation were evaluated for analytical techniques as below Table 2:

TABLE 2
Stability Study Plan

Time Point (Days) for DP

| Analytical Test | 0 D | 14 D | 28 D |
|---|---|---|---|
| Physical Appearance | X | X | X |
| Reconstitution Time | X | X | X |
| Physical Appearance after | X | X | X |
| Reconstitution | | | |
| pH | X | X | X |
| Protein Concentration | X | X | X |
| Pro-A Oxidation | X | X | X |
| CEX-HPLC | X | X | X |
| SEC-HPLC | X | X | X |
| Relative Potency | X | NA | X |
| CE-SDS (R & NR) | X | X | X |
| CIEF | X | X | X |

X: Testing Time Point
NA: Not Applicable

### Example 2: Stress Stability Data of Formulations (Lyophilized)

### A) Physical Appearance Before and After Reconstitution:

All the samples were observed to be white to White to off-white lyophilized cake before reconstitution and after reconstitution all the samples were observed clear & colorless to light brownish yellow color liquid.

### B) pH:

TABLE 1
pH of RMP, F1, F2, F3, F4, F5, F6, F7, F8, F9, F10, & F11

| | pH | | |
|---|---|---|---|
| Buffers | 0 D | 14 D | 28 D |
| RMP | 6.27 | Not Tested | 6.21 |
| F1 | 6.47 | 6.54 | 6.44 |
| F2 | 6.55 | 6.58 | 6.51 |
| F3 | 6.54 | 6.52 | 6.56 |
| F4 | 6.55 | 6.54 | 6.49 |
| F5 | 6.56 | 6.52 | 6.47 |
| F6 | 6.6 | 6.58 | 6.54 |
| F7 | 6.58 | 6.54 | 6.53 |
| F8 | 6.55 | 6.58 | 6.5 |
| F9 | 6.58 | 6.59 | 6.53 |
| F10 | 6.3 | 6.3 | 6.3 |
| F11 | 6.26 | 6.27 | 6.27 |

Observation: Based on 28 days stress data the pH of the F10 and F11 was comparable with RMP for pH.

### C) CEX-HPLC

TABLE 2
CEX data of RMP, F1, F2, F4, F5, F6, F7, F8, F9, F10 & F11

| | % Purity | | |
|---|---|---|---|
| Buffers | 0 D | 14 D | 28 D |
| RMP | 73.3 | Not Tested | 46.4 |
| F1 | 51 | 40.7 | 35 |
| F2 | 50.2 | 41.3 | 36.4 |
| F4 | 51 | 42 | 37.4 |
| F5 | 50.3 | 41.5 | 38 |
| F6 | 50.7 | 42.6 | 36.5 |
| F7 | 50.5 | 42.3 | 37.8 |
| F8 | 50.5 | 44.7 | 38.9 |
| F9 | 50.5 | 43.1 | 37.1 |
| F10 | 58.7 | 59.5 | 56 |
| F11 | 58.9 | 59.3 | 55.7 |

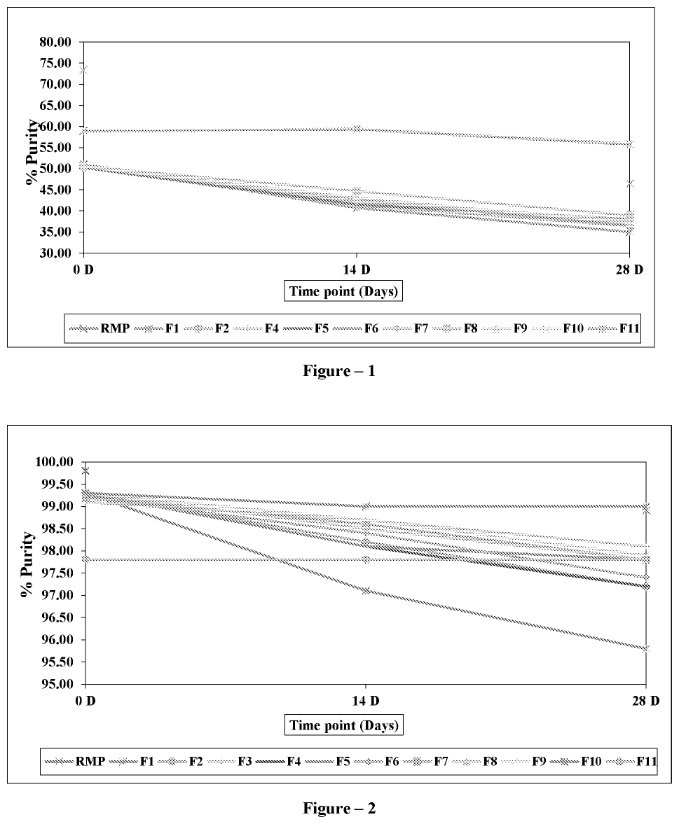

Observation: Based on 28 Days stress data the purity of the F10 and F11 was comparable with RMP. As also shown by FIG. 1.

### D) SEC-HPLC

TABLE 3
SEC data of RMP, F1, F2, F3, F4, F5, F6, F7, F8, F9, F10 & F11

| | % Purity | | |
|---|---|---|---|
| Buffers | 0 D | 14 D | 28 D |
| RMP | 99.8 | Not Tested | 98.9 |
| F1 | 99.3 | 97.1 | 95.8 |
| F2 | 99.3 | 98.2 | 97.2 |
| F3 | 99.1 | 98.7 | 98.1 |
| F4 | 99.3 | 98.1 | 97.2 |
| F5 | 99.3 | 98.1 | 97.8 |
| F6 | 99.2 | 98.4 | 97.4 |
| F7 | 99.2 | 98.6 | 97.8 |
| F8 | 99.3 | 98.5 | 97.8 |
| F9 | 99.3 | 98.7 | 97.9 |
| F10 | 99.3 | 99 | 99 |
| F11 | 97.8 | 97.8 | 97.8 |

Observation: Based on 28 Days stress data the purity of the F10 and F11 was comparable with RMP. As also shown by FIG. 2.

### Example 3: Real Time (2-8° C.) Stability Data of Batch 1 (P53F122D86U115), Batch 2 (P53F124D88U131) and Batch 3 (P53F126D85U102)

Three different consistency batches BATCH 1 (P53F122D86U115), BATCH 2 (P53F124D88U131) AND BATCH 3 (P53F126D85U102) of formulation 11 (F11) was prepared to assess the real time stability. Following is the result from the real time (2-8° C.) stability study.

### A) Physical Appearance:

All the samples were observed to be white to White to off-white lyophilized cake before reconstitution and after reconstitution all the samples were observed clear & colorless to light brownish yellow color liquid.

### B) pH:

TABLE 4
pH data of Batch 1, 2 & 3

| | |
|---|---|
| Buffers | pH |

| Time Points | 0 D | 1 M | 2 M | 3 M | 6 M |
|---|---|---|---|---|---|
| Batch 1 | 6.34 | 6.26 | 6.28 | 6.28 | 6.34 |
| Batch 2 | 6.32 | 6.26 | 6.27 | 6.29 | 6.34 |
| Batch 3 | 6.45 | 6.40 | 6.38 | 6.42 | 6.48 |

Observation: Based on 6 months real time data the pH of all the 3 batches were comparable.

### C) CEX-HPLC

TABLE 5
CEX data of Batch 1, 2 & 3

| | |
|---|---|
| Buffers | % Purity CEX |

| Time Points | 0 D | 1 M | 2 M | 3 M |
|---|---|---|---|---|
| Batch 1 | 67.5 | 66.9 | 64.4 | 64.0 |
| Batch 2 | 66.4 | 67.7 | 67.3 | 64.7 |
| Batch 3 | 65.4 | 66.3 | 65.1 | 62.3 |

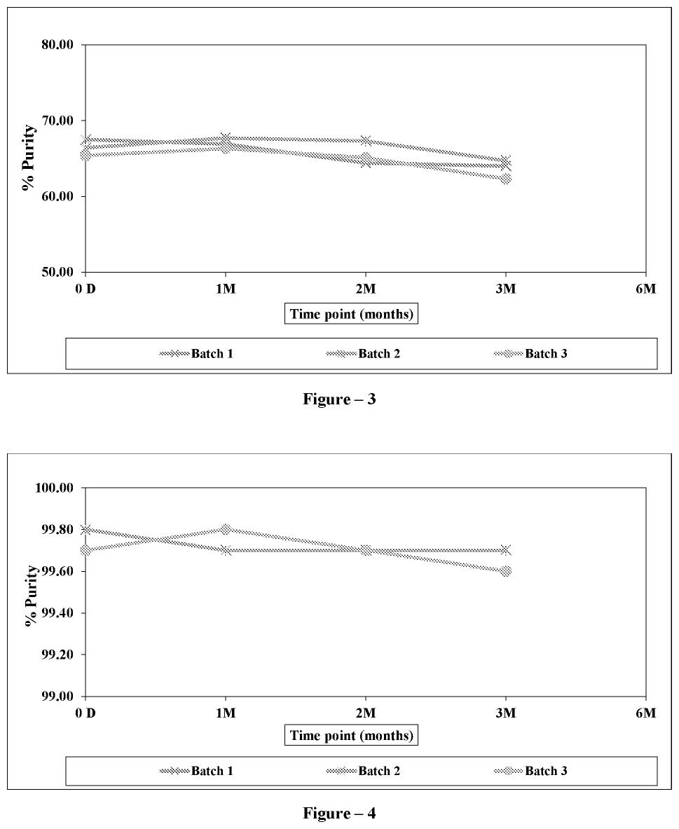

Observation: Based on 3 months real time data the purity of all the 3 batches were comparable. As also shown by FIG. 3.

### D) SEC-HPLC

TABLE 6
SEC data of Batch 1, 2 & 3

| | |
|---|---|
| Buffers | % purity SEC |

| Time Points | 0 D | 1 M | 2 M | 3 M |
|---|---|---|---|---|
| Batch 1 | 99.8 | 99.7 | 99.7 | 99.7 |
| Batch 2 | 99.8 | 99.7 | 99.7 | 99.7 |
| Batch 3 | 99.7 | 99.8 | 99.7 | 99.6 |

Observation: Based on 3 months real time data the purity of all the 3 batches were comparable. As also shown by FIG. 4.

### E) Potency:

The biological activity of Vedolizumab is determined by cell based in vitro bio-assay. RPMI8866 cell line is used as a source of α4β7 receptor. The assay is based on dose dependent inhibition of binding of a487 expressed on RPMI8866 with MAdCAM-1. In presence of Vedolizumab, cell adhesion to MAdCAM-1 is inhibited in a dose dependent manner.

TABLE 7
% Potency data of Batch 1, 2 & 3

| | |
|---|---|
| Buffers | Relative potency |

| Time Points | 0 D | 1 M | 2 M | 3 M | 6 M |
|---|---|---|---|---|---|
| Batch 1 | 106 | 86 | 93 | 90 | 87 |
| Batch 2 | 114 | 93 | 108 | 117 | 98 |
| Batch 3 | 115 | 96 | 106 | 97 | 93 |

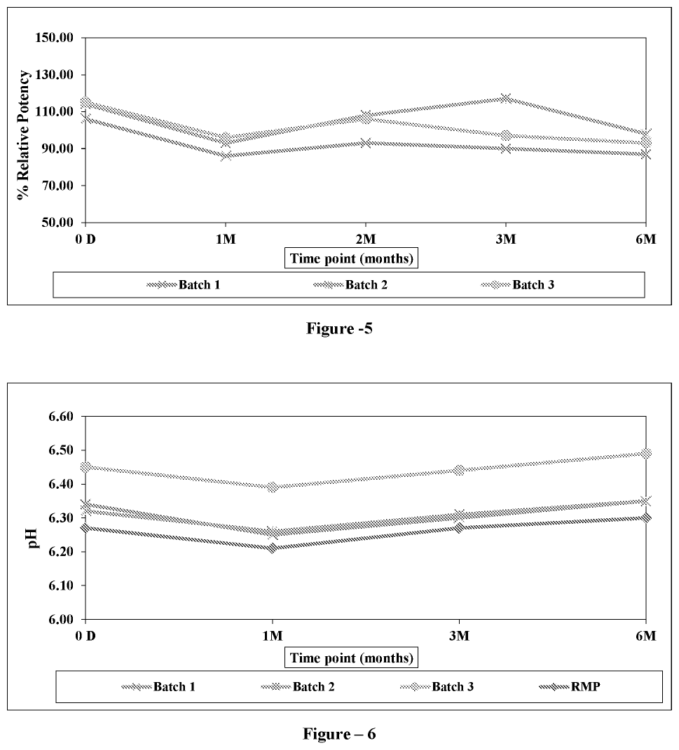

Observation: There is no change in potency at 2-8° C. after 6 months as compared to initial in all 3 batches. As also shown by FIG. 5.

### Example 4: Accelerated (25° C.) and Stress Stability (40° C.) Data of Batch 1, Batch 2 and Batch 3

Three different consistency batches (same as example 2) BATCH 1 (P53F122D86U115), BATCH 2 (P53F124D88U131) AND BATCH 3 (P53F126D85U102) of formulation 11 (F11) was used to assess the accelerated stability. Following is the result from the accelerated (25° C.) stability study.

### A) Physical Appearance:

All the samples were observed to be white to white to off-white lyophilized cake before reconstitution and after reconstitution all samples were observed clear & colorless to light brownish yellow liquid.

### B) pH:

TABLE 8
pH data of Batch 1, 2 & 3 at AT (25° C.)

| | |
|---|---|
| Buffers | pH |

| Time Points | 0 D | 1 M | 3 M | 6 M |
|---|---|---|---|---|
| Batch 1 | 6.34 | 6.25 | 6.30 | 6.35 |
| Batch 2 | 6.32 | 6.26 | 6.31 | 6.35 |
| Batch 3 | 6.45 | 6.39 | 6.44 | 6.49 |
| RMP | 6.27 | 6.21 | 6.27 | 6.30 |

Observation: Based on 6 months accelerated stability data the pH of all the 3 batches were comparable with the reference formulation (RMP). As also shown by FIG. 6.

TABLE 9
pH of Batch 1, 2 & 3 at ST (40° C.)

| Buffers | pH | | |
|---|---|---|---|
| Time Points | 0 D | 14 D | 28 D |
| Batch 1 | 6.34 | 6.27 | 6.29 |
| Batch 2 | 6.32 | 6.29 | 6.28 |
| Batch 3 | 6.45 | 6.42 | 6.42 |
| RMP | 6.27 | 6.23 | 6.23 |

Observation: Based on 28 days stress data, the pH of all the 3 batches were comparable with the reference formulation (RMP). As also shown by FIG. 7.

### C) CEX-HPLC

TABLE 10
CEX data of Batch 1, 2 & 3 at AT (25° C.)

| Buffers | % Purity | | |
|---|---|---|---|
| Time Points | 0 D | 1 M | 3 M |
| Batch 1 | 67.5 | 66.6 | 62.8 |
| Batch 2 | 66.4 | 67.5 | 64.7 |
| Batch 3 | 65.4 | 65.4 | 60.0 |
| RMP | 70.5 | 71.1 | 70.4 |

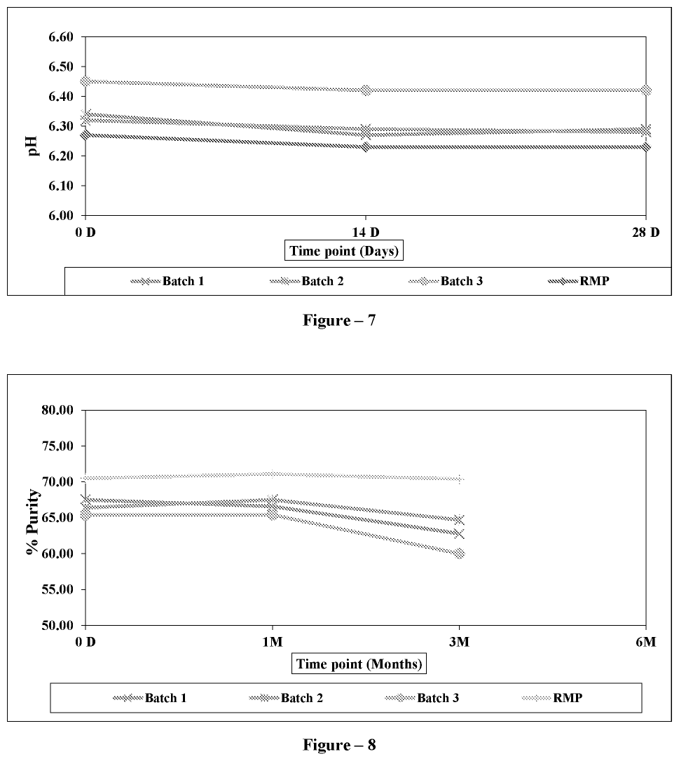

Observation: Based on 3 months accelerated stability data the purity of all the 3 batches were comparable with the reference formulation (RMP). As also shown by FIG. 8.

TABLE 11
CEX data of Batch 1, 2 & 3 at ST (40° C.)

| Buffers | % Purity | | |
|---|---|---|---|
| Time Points | 0 D | 14 D | 28 D |
| Batch 1 | 67.5 | 65.8 | 65.0 |
| Batch 2 | 66.4 | 66.9 | 64.7 |
| Batch 3 | 65.4 | 64.2 | 64.1 |
| RMP | 70.5 | 68.9 | 69.5 |

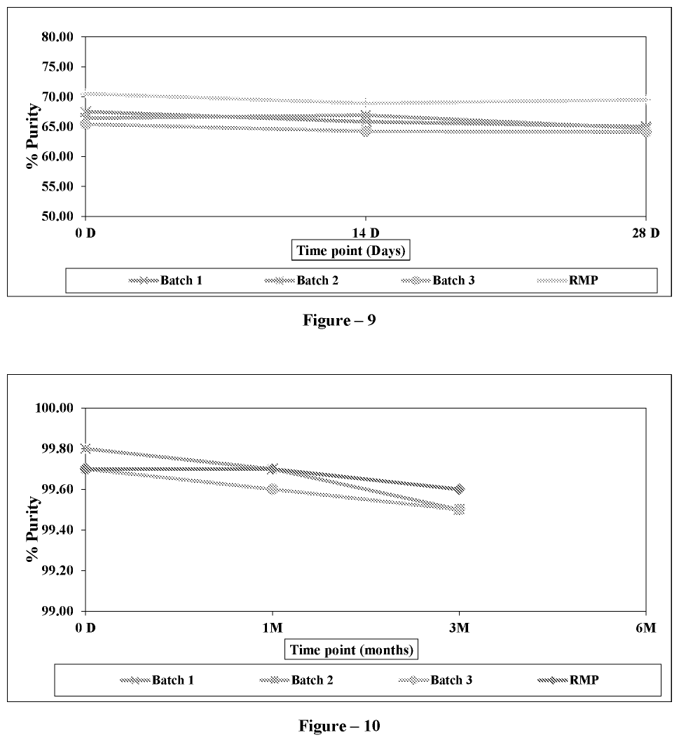

Observation: Based on 28 days stress data, the purity of all the 3 batches was comparable with the reference formulation (RMP). As also shown by FIG. 9.

### F) SEC-HPLC

TABLE 12
SEC data of Batch 1, 2 & 3 at AT (25° C.)

| Buffers | % Purity SEC | | |
|---|---|---|---|
| Time Points | 0 D | 1 M | 3 M |
| Batch 1 | 99.8 | 99.7 | 99.5 |
| Batch 2 | 99.8 | 99.7 | 99.5 |
| Batch 3 | 99.7 | 99.6 | 99.5 |
| RMP | 99.7 | 99.7 | 99.6 |

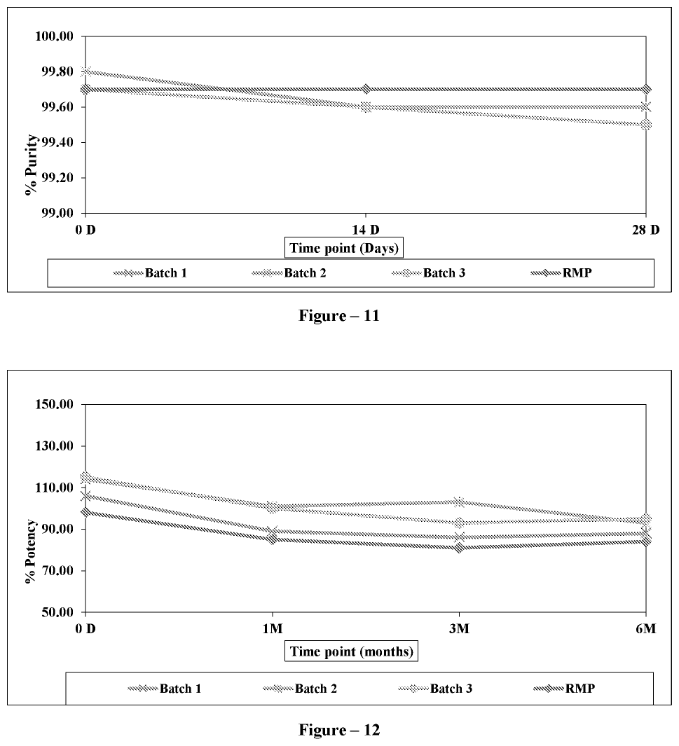

Observation: Based on 3 months accelerated stability data the purity of all the 3 batches were comparable with the reference formulation (RMP). As also shown by FIG. 10.

TABLE 13
SEC data of Batch 1, 2 & 3 at ST (40° C.)

| Buffers | % Purity SEC | | |
|---|---|---|---|
| Time Points | 0 D | 14 D | 28 D |
| Batch 1 | 99.8 | 99.6 | 99.6 |
| Batch 2 | 99.8 | 99.6 | 99.6 |
| Batch 3 | 99.7 | 99.6 | 99.5 |
| RMP | 99.7 | 99.7 | 99.7 |

Observation: Based on 28 days stress data, the purity of all the 3 batches were comparable with the reference formulation (RMP). As shown by FIG. 11.

### D) Potency:

The biological activity of Vedolizumab is determined by cell based in vitro bio-assay. RPMI8866 cell line is used as a source of a487 receptor. The assay is based on dose dependent inhibition of binding of α4β7 expressed on RPMI8866 with MAdCAM-1. In presence of Vedolizumab, cell adhesion to MAdCAM-1 is inhibited in a dose dependent manner.

TABLE 14
% Potency data of Batch 1, 2 & 3 at AT (25° C.)

| | |
|---|---|
| Buffers | Relative potency |

| Time Points | 0 D | 1 M | 3 M | 6 M |
|---|---|---|---|---|
| Batch 1 | 106 | 89 | 86 | 88 |
| Batch 2 | 114 | 101 | 103 | 93 |
| Batch 3 | 115 | 100 | 93 | 95 |
| RMP | 98 | 85 | 81 | 84 |

Observation: There is no change in potency at 25° C. after 6 months as compared to initial in all 3 batches. As also shown by FIG. 12.

TABLE 15
% Potency data of Batch 1, 2 & 3 at ST (40° C.)

| Buffers | Relative potency | | |
|---|---|---|---|
| Time Points | 0 D | 14 D | 28 D |
| Batch 1 | 106 | NA | 90 |
| Batch 2 | 114 | NA | 104 |
| Batch 3 | 115 | NA | 100 |
| RMP | 98 | NA | 84 |

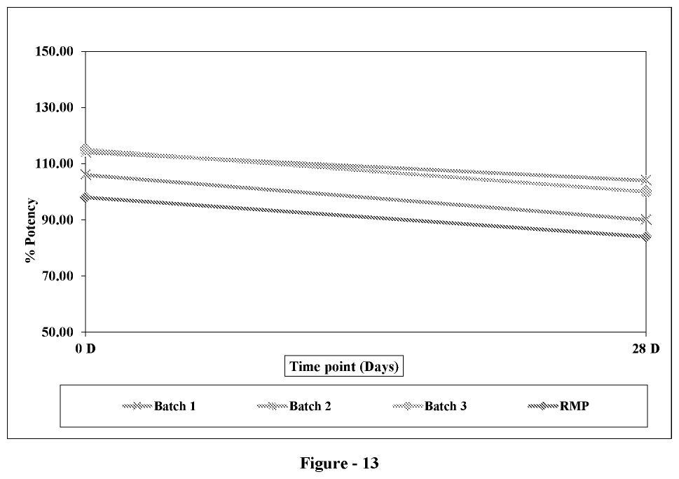

Observation: There is no change in potency at 40° C. after 28 days as compared to initial in all 3 batches. As also shown by FIG. 13.

Results from the % potency data were significant since it indicates that there is no change in terms of potency between formulation of the present invention and RMP formulation. This means that formulation of the present invention is highly comparable to RMP.

We claim:

1. A stable lyophilized pharmaceutical formulation of an anti-α4ß7 antibody comprising: a buffer, sugar, bulking agent, viscosity reducer, antioxidant and surfactant.

2. The lyophilized pharmaceutical formulation of claim 1, wherein the bulking agent is present in the concentration of 0-50 mg/ml, preferably about 15-30 mg/ml.

3. The lyophilized pharmaceutical formulation of claim 1, wherein the viscosity reducer is present in the concentration of 0-10 mg/ml, preferably about 4-8 mg/ml.

4. The lyophilized pharmaceutical formulation of claim 1, wherein the antioxidant is present in the concentration of 0-2 mg/ml, preferably about 0.75 mg/ml.

5. The lyophilized pharmaceutical formulation of claim 1, wherein the bulking agent is glycine, viscosity reducer is L-lysine HCl and antioxidant is L-methionine.

6. The lyophilized pharmaceutical formulation of claim 1, wherein the molar ratio of sugar to antibody (mole:mole) is less than 600:1.

7. The lyophilized pharmaceutical formulation of claim 1, wherein the buffer is histidine, sugar is sucrose and surfactant is polysorbate 80.

8. A lyophilized pharmaceutical formulation of Vedolizumab comprising: histidine and histidine HCl as buffer, sucrose as stabilizer, glycine as bulking agent, L-lysine HCl as viscosity reducer, L-methionine as antioxidant, polysorbate 80 as surfactant and at pH about 6.0 to 7.0, wherein the molar ratio of Sucrose to Vedolizumab (mole:mole) is less than 600:1.

9. The lyophilized pharmaceutical formulation of claim 8, wherein the formulation comprises: 0-100 mM histidine/histidine HCl, 0-100 mg/mL sucrose, 0-50 mg/mL glycine, 0-10 mg/mL L-lysine HCl, 0-2 mg/mL L-methionine, 0-1 mg/mL polysorbate 80 and at pH about 6.0 to 7.0.

10. The lyophilized pharmaceutical formulation of claim 8, wherein the formulation comprises 60 mg/mL Vedolizumab, 50 mM histidine/histidine HCL, 80 mg/mL sucrose, 30 mg/mL glycine, 8 mg/mL L-lysine HCl, 0.75 mg/mL L-methionine, 0.6 mg/mL polysorbate 80 at pH 6.3.
